(19) United States
(12) Patent Application Publication
(10) Pub. No.: US 2008/0202215 A1
(43) Pub. Date: Aug. 28, 2008

(54) PRESSURIZED CRYSTALLIZATION POINT AUTOMATED TEST APPARATUS

(75) Inventors: Kenneth Slater, Houston, TX (US); Marian Baranowski, Houston, TX (US); Arkadiy Belkin, Houston, TX (US)

Correspondence Address: LEGAL DEPT., IP GROUP;M-I L.L.C., 5950 NORTH COURSE DRIVE, HOUSTON, TX, 77072, US

(73) Assignee: M-I L.L.C., Houston, TX (US)

(21) Appl. No.: 11/678,040

(22) Filed: Feb. 22, 2007

(51) Int. Cl. G01N 33/00; B01L 11/00

(52) U.S. Cl. 73/53.01

(57) ABSTRACT

A pressurized crystallization point test apparatus for pressurizing a fluid sample and optically detecting the degree of crystallization in the fluid sample while simultaneously measuring the temperature and change in volume of the fluid sample during cooling or heating of the sample is disclosed. The apparatus includes: a cell for containing a fluid sample in an interior of the cell, wherein the cell comprises a first transparent window and a second transparent window; a cooling source for cooling the fluid sample; a pressure source for pressurizing the fluid sample; a temperature sensor positioned in the interior of the cell for measuring the temperature of the fluid sample; an external light source configured to direct light into the cell through the first transparent window; and an external light detector configured to measure the amount of light that traverses the first transparent window, a portion of the fluid sample, and the second transparent window.

### BACKGROUND

Brine fluids are commonly used as completion, workover, and drilling fluids during subterranean well operations. Brines are aqueous solutions of one or more salts. The salts are typically chlorides, bromides, or formates such as sodium chloride, calcium chloride, calcium bromide, potassium chloride, potassium formate, and sodium formate to name a few. Brines are formulated with a salt density typically in a range from about 8 to about 20 lb/gal depending on the particular use and specific conditions. Brines are commonly used for pressure control because of their non-damaging character as solids free solutions that contain no particles that may damage or plug a producing formation. As such, the density and crystallization temperature of a brine are important specified parameters in normal industry practice.

It is well known that the use of brines in low temperature or high pressure conditions presents a problem of brine crystallization. At temperatures at or below the crystallization temperature of the brine, the precipitation of crystallizing solids (e.g., salts) can change the density of the brine fluid and deteriorate the ability of the fluid to maintain pressure control.

Brine crystallization may also lead to crystallized solids plugging the filters and lines in the subterranean well. In addition, brines under high pressure conditions experience a shift in the crystallization temperature of the brine to a higher temperature than expected under ambient pressure conditions.

Most notably, the combination of low temperature and high hydrostatic pressure conditions encountered at the sea floor during deep sea well operations presents a problem of increasing the crystallization temperatures of brines. Thus the crystallization temperature of a pressurized brine fluid is an essential parameter to know for low temperature and high pressure applications. For a more thorough discussion regarding the effect of pressure on crystallization temperature refer to a technical paper by Michael A. Freeman et al., entitled “High Pressure Crystallization of Deep-Water Completion Brines,” SPE 58729, presented at the 2000 SPE International Symposium on Formation Damage held in Lafayette, La., Feb. 23-24, 2000, incorporated herein by reference.

The crystallization temperature of a brine at ambient pressure is commonly measured in accordance to a standardized test method described in ANSI/API Recommended Practice 13J, entitled “Testing of Heavy Brines”, 4^{th} Ed. (May 2006). To characterize the crystallization profile of the brine, as described in API Recommended Practice 13J, an apparatus is used to alternately cool and heat a sample of brine fluid for measuring three different crystallization temperatures. During testing, the sample is slowly and continuously cooled until a temperature is reached at which visible crystals start to form in the sample and the temperature is recorded as the First Crystal to Appear (FCTA) temperature. During cooling, the FCTA temperature corresponds to a minimum inflection point in a plot of temperature versus time, the minimum inflection point being generally the result of a super-cooling effect. Upon reaching the FCTA temperature, the cooling temperature is held constant while the exothermic brine crystallization process proceeds. Heat is released during the brine crystallization process and the maximum temperature, or maximum inflection point, reached immediately following the FCTA temperature is recorded as the True Crystallization Temperature (TCT). The TCT corresponds to the actual true crystallization temperature of the brine. After obtaining the TCT, cooling is discontinued and the brine is allowed to warm, or is heated, to dissolve the crystals. The temperature at which the last crystal is observed to disappear is recorded as the Last Crystal to Dissolve (LCTD) temperature.

The LCTD temperature also corresponds to a minimum inflection point due to an increase in the heating rate of the brine just after the crystals have completely dissolved.

According to ANSI/API Recommended Practice 13J, it is recommended that the cooling/heating testing described above is repeated at least three times for a given brine sample and the average measurements are reported as the FCTA, TCT, and LCTD temperatures for the brine. The accuracy of the FCTA, TCT, and LCTD measurements is, in part, affected by the rate of cooling, rate of heating, and visual observation of crystallization.

Visual inspection of the brine sample during testing enhances accuracy because one or more of the crystallization event inflection points on a temperature versus time plot is often subtle and difficult to identify.

The apparatus of the present invention provides an automated pressurized crystallization point test apparatus as an alternative apparatus to those described in the prior art. In particular, one such apparatus described in the prior art uses a fiberoptic probe for optically detecting crystallization under pressurized conditions. In the preferred configuration, a fiberoptic probe with a closely-spaced mirror is immersed in a sample solution to detect crystals across a small portion of the sample solution. Optically examining only a small volume of the sample is undesirable in that it limits the accuracy particularly with respect to detecting the first crystal to appear and the last crystal to dissolve during FCTA and LCTD measurements. Another disadvantage of utilizing an immersed fiberoptic probe is the potential for fouling the tip of the probe and/or mirror submerged in the pressurized sample solution, which may adversely affect accuracy and reproducibility of measurements. Additionally, the presence of the immersed probe undesirably interferes with the circulation of the sample during stirring. In addition to these disadvantages or limitations, is the relatively expensive cost of a fiberoptic probe.

Despite efforts in the prior art, there is a need for an automated apparatus and method that provides highly accurate and reproducible crystallization temperature measurements of fluids under pressurized conditions.

### SUMMARY

The subject matter of the present disclosure is generally directed to an apparatus for measuring the crystallization temperatures of a fluid under pressurized conditions. The present invention provides an automated apparatus having an optical capability for detecting crystals in the fluid sample, thus eliminating the need for a person to visually observe the sample for the presence of crystals when measuring the crystallization events FCTA, TCT, and LCTD of a brine sample. The apparatus is also equipped with a pressure source for pressurizing the fluid sample to high pressures for simulating pressure conditions encountered during subsea well operations. Another advantage of the present invention is that the optical technique employed enhances the accuracy in determining the FCTA and LCTD temperatures due to its high sensitivity in detecting crystals in solution. Furthermore, the optical technique detects crystallization in a sufficient volume of the brine sample which allows for higher measurement accuracy and reproducibility particularly with respect to detecting the first crystal and last crystal to dissolve temperature measurements FCTA and LCTD.

The apparatus of the invention comprises: a cell for containing a fluid sample in an interior of the cell, wherein the cell comprises a first transparent window and a second transparent window; a cooling source for cooling the fluid sample; a pressure source for pressurizing the interior of the cell; a temperature sensor positioned in the interior of the cell for measuring the temperature of the fluid sample; an external light source configured to direct light into the cell through the first transparent window; and an external light detector configured to measure the amount of light that traverses the first transparent window, a portion of the fluid sample within the cell, and the second transparent window.

These and other features are more fully set forth in the following description of preferred or illustrative embodiments of the disclosed and claimed subject matter.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustratively depicts a partial cross-sectional view of one embodiment of the apparatus of the present invention.

FIG. 2 illustratively depicts a partial cross-sectional view of another embodiment of the apparatus having a variable-volume isolation vial.

FIG. 3 illustratively depicts a partial cross-sectional view of still another embodiment of the apparatus incorporating a cooling-magnetic stir-plate.

### DETAILED DESCRIPTION

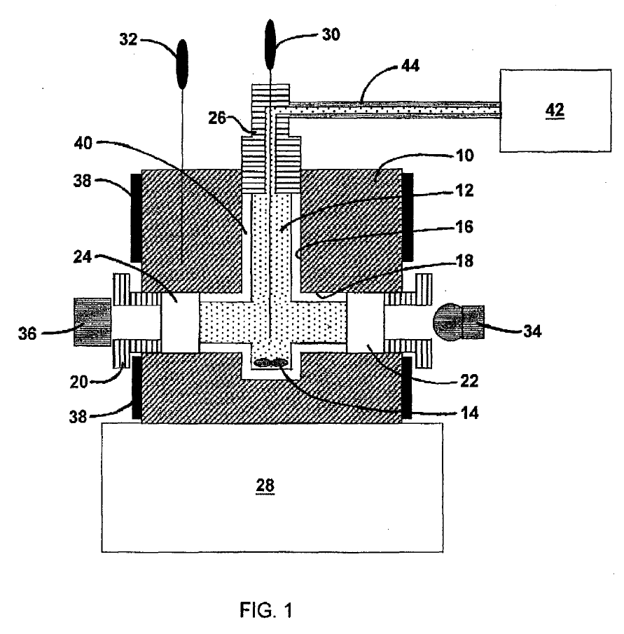

FIG. 1 is a schematic illustration of an embodiment of the apparatus of the present invention equipped with optical instrumentation and a pressure source to carry out a test method for determining the crystallization events of a pressurized fluid. The apparatus comprises a cell 10 for containing a brine fluid sample 12 and a magnetic stir-pill 14 therein. The body of the cell 10 is preferably a three opening “Tee”-style fitting adapted for high pressure applications. As depicted, a vertical aperture 16 and a horizontal aperture 18 constitute the internal cavity of the cell 10. One example of a suitable cell 10 is a stainless steel Tee-style fitting rated for internal cavity pressures up to 20,000 psi, commercially available from High Pressure Equipment Co. (Cat. No. 20-23LF16), Erie, Pa., however other cell designs suitable for high pressure applications might alternatively be used.

The cell 10 is made of thermally conductive material for the purpose of transferring heat to or from the sample 12 during testing. Suitable cell 10 materials include metals, metal alloys, or other thermally conductive materials that can withstand the desired testing temperatures and pressures of the fluid sample 12. Threaded glands 20 inserted at two openings of the cell 10 are used to position and secure a first transparent window 22 and a second transparent window 24 within the horizontal aperture 18 of the cell 10. The windows 22, 24 may be positioned anywhere along the horizontal aperture 18. Windows 22, 24 should be transparent and optically clear to enable light to pass through the windows 22, 24. Windows 22, 24 may be made of glass, quartz, plastic, or other optically clear material that can withstand the desired testing temperatures and pressures of the fluid sample 12. A cap 26, and windows 22, 24 contain the brine sample 12 within an interior central cavity of the cell 10. The volume capacity of the cell 10 is preferably selected to accommodate the desired testing volume of the brine sample 12 within the interior central cavity of the cell 10. The testing volume of the brine fluid sample 12 is typically in a range of about 25 ml to about 75 ml, however any quantity may be used. Furthermore, the cell 10 containing the magnetic stir-pill 14 is positioned above a magnetic stirring plate 28 for stirring the brine sample 12 during testing. However, stirring the sample 12 may be accomplished by other stirrers or stirring systems that should be well known to one of skill in the art.

A temperature sensor 30 extends into the brine fluid sample 12 to measure the temperature of the brine sample 12 during testing. Preferably the temperature sensor 30 is a RTD probe, however other temperature sensors, for example, a thermocouple or a thermometer, may be used. The temperature sensor 30 may be mounted to an interior surface of the cap 26, or otherwise attached to the apparatus, such that it extends into the brine fluid sample 12. Another temperature sensor 32 (e.g., a RTD probe) attached to the cell 10 is used to measure the temperature of the cell 10 to aid in its temperature control.

To optically detect the presence of crystals in the brine sample 12, the apparatus is equipped with optical instrumentation comprising an external light source 34 and an external light detector 36. The light source 34 and light detector 36 are externally positioned outside the interior central cavity of the cell 10, and adjacent the first and second transparent windows 22, 24, respectively, such that there is no contact with the brine sample 12. The light source 34 and light detector 36 are laterally spaced such that the path of a light beam emanating from the light source 34 is directed into the interior central cavity of the cell 10 and towards the light detector 36. A suitable light source includes light emitters such as lasers, lamps, light emitting diodes (LEDs), or any other light emitter that can transmit light across the windows 22, 24 and into the light detector 36. The light emanating from the light source 34 may be essentially any type of light including visible, polarized, laser, IR, and UV. Likewise, suitable light detectors include a photo-resistor, photo-transistor, photodiode, photovoltaic cell, and other detectors that should be familiar to one of skill in the art.

Furthermore, the optical instrumentation is preferably configured such that the light beam may travel in a single pass from the light source 34 through the first transparent window 22, through a portion of the brine sample 12 within the cell 10, and then through the second transparent window 24 and into the light detector 36. This configuration allows for detection of crystals in the portion of the brine sample through which the light travels as the light traverses the cell 10. Thus, while the brine sample 12 is continuously stirred during testing, the portion of the brine sample that is optically detectable is the constant volume of the brine sample in the path of the light beam. The portion of the brine sample 12 optically monitored should be a sufficient volume for providing high accuracy in optically detecting the presence of crystals in fluids containing only a very dilute concentration of crystals, for example when detecting the FCTA and LCTD temperatures. For typical 25 ml to 75 ml brine samples, the portion of the brine sample 12 optically monitored is preferably equal to a volume of about 5% or more of the total brine sample volume. More preferably, the portion of the brine sample 12 optically monitored is a volume in a range from about 10% to about 30% of the total brine sample volume. Thus, for ensuring high accuracy in detecting the degree of crystallization in dilute crystal solutions, the optical instrumentation should be configured to optically monitor at least about 2 ml of the brine sample 12, and more preferably from about 4 ml to about 10 ml of the brine sample 12. In general, increasing the amount of volume optically monitored increases the accuracy of the optical technique in detecting the first crystal to appear and the last crystal to dissolve temperature measurements FCTA and LCTD.

During testing, the light detector 36 detects the presence of crystals by continually measuring the light transmission across the cell 10 and the portion of the brine sample 12 in the path of the light beam. When there are no crystals in the brine fluid sample 12, the emitted beam travels through the cell 10 and optically-clear brine sample 12 and is received by the light detector 36 (e.g., photo-resistor). During cooling, when crystals form in the brine sample 12, light is blocked by the opaque crystals thus reducing the amount of the light beam that passes completely through the sample 12 and into the light detector 36. The attenuated light detected by the light detector 36 is related to the degree of crystallization in the brine sample 12. This optical technique is highly sensitive to detecting crystals in solution. By detecting crystallization across at least about 2 ml of the brine sample 12, this optical technique provides very accurate and reproducible detection of crystallization events FCTA and LCTD.

It should be noted that the present invention does not limit the position of the light detector 36 to a position within the straight path of the light beam, as depicted in FIG. 1. While the light detector 36, positioned in the straight path of the light beam, measures a decrease in light as crystals form in the brine sample 12, conversely, another light detector (not shown) may be used that is positioned, for example, orthogonal to the straight path of the light beam which measures an increase in light due to the scattering of the light blocked by the opaque crystals. Thus, an increase in light detected by the orthogonally-positioned light detector is related to an increase in crystallization of the brine sample. One example of a cell that may accommodate another light detector positioned orthogonal to the straight path of the light beam is a stainless steel 4 opening “Cross”-style fitting rated for internal cavity pressures up to 20,000 psi, commercially available from High Pressure Equipment Co. (Cat. No. 20-24LF16), Erie, Pa., however other cell designs suitable for high pressure applications might alternatively be used.

As depicted in FIG. 1, Peltier junctions 38 provide the cooling source for cooling the cell 10 and brine fluid sample 12 therein. The Peltier junction is a thermoelectric cooling device having a cold ceramic plate on one side and a hot ceramic plate on the other side. While heat is drawn away from the cold plate to the hot plate, the hot plate must be cooled. Accordingly, the cold plate side of each of the Peltier junctions 38 contacts the cell 10, for cooling the cell 10, and the hot plate side contacts a water jacket (not shown) to dissipate heat drawn to the hot plate side. The water jacket circulates tap water, or room-temperature water, to sufficiently cool the hot plate sides of the Peltier junctions 38. The temperature control of the Peltier junctions 38 is achieved by controlling the efficiency at which heat is removed from the hot plate sides. The relevant and controllable parameters include the flow rate and temperature of the water circulating through the water jacket, as well as regulating the power to the Peltier junctions 38. The water-cooled Peltier junctions 38 supply adequate cooling to the cell 10 for crystallization testing while also providing cooling that is both environmentally safe and portable for on-site or field use of the apparatus.

Cooling of the brine sample 12 may be accomplished by alternative cooling systems that should be familiar to one of skill in the art. For example, instead of using Peltier junctions 38, a cooling jacket (not shown) that circulates chilled water (or coolant) could be placed in contact with, or in close proximity to, the cell 10 for controlling its temperature. In another example, a cooling bath (not shown) that circulates chilled water, ice, or coolant around the cell 10 may also be used for cooling. In another example, a refrigerator may be used to cool the cell 10 by placing the apparatus in a temperature-controlled refrigerator. Any cooling system or combination of cooling systems may be used to cool the cell 10.

A liner 40 along the surface of the interior central cavity of the cell 10 is optionally used to insulate and slow the cooling of the brine fluid sample 12. At one point during the test when cooling is stopped, the liner 40 sufficiently slows the heat transfer from the sample 12 such that the heat generated during the exothermic crystallization effectuates a rise in temperature for TCT measurement. Without the liner 40, the heat generated during crystallization may be drawn away from the sample 12 too quickly, thus making detection of a rise in temperature more difficult. The liner 40 is preferably made of an insulating material, such as Teflon® or other plastic.

The apparatus is also equipped with a pressure source 42 for pressurizing the brine fluid sample 12 during testing. The pressure source is preferably a pressure intensifier capable of pressurizing the brine sample 12 to a pressure in the range of about 0 psi to about 20,000 psi. One example of a pressure intensifier is an air-actuated pressure intensifier wherein air supplied to the inlet of the pressure intensifier forces the positive displacement piston, having an area ratio of about 16:1, to apply the desired force to the brine at the outlet of the pressure intensifier. The pressure source 42 provides pressurized brine in a pressurized line 44 and to the interior central cavity of the cell 10. The pressure source provides continuous control over the pressure applied to the brine sample 12 throughout the test. Utilizing a pressure source having a positive displacement piston advantageously allows for monitoring the change in volume of the brine sample 12 during testing. Typically, as the brine sample 12 cools it experiences a reduction in volume, and during crystallization events there is usually a measurable change in the rate of volume reduction that may be monitored as another means of detecting the crystallization events of the brine sample 12. Depending upon the desired testing conditions, the pressure source may provide pressurized brine having a pressure in excess of 20,000 psi. Also, pressurization of the brine sample 12 may be accomplished by alternative pressurizing systems that should be familiar to one of skill in the art.

The apparatus may optionally comprise an insulating cover (not shown) that surrounds the outer surfaces of the cell 10 to enhance temperature control of the cell 10. Although the particular configuration of the insulating cover is not important, providing insulation surrounding any otherwise exposed outer surfaces of the cell 10 is preferable in order to enhance temperature uniformity throughout the cell 10. The insulating cover may be made of any thermally non-conductive material, such as fiberglass, foam or other polymeric material.

Furthermore, the apparatus is connected to a computer (not shown) to automate the test procedure for testing in a manner consistent with the procedure described in ANSI/API Recommended Practice 13J. During the initial set-up, the computer controller software is initialized by providing the cooling and heating rates, the desired testing pressure, a set-point hold temperature above LCTD, and event triggering levels of the light detector.

The computer control is designed to automatically adjust the pressure source 42 to maintain the desired constant testing pressure of the brine sample 12. The computer control also automatically adjusts the cooling and heating of the brine sample 12 throughout the test. During the test, the computer monitors the real-time temperature of the brine sample 12, the temperature of the cell 10, the volume change of the brine sample 12, and the light detector's 36 light attenuation data (i.e., degree of crystallization data). The light attenuation data and the temperature of the brine sample 12 are used to automate the proper control over the cooling and heating rates of the brine sample 12 throughout the test.

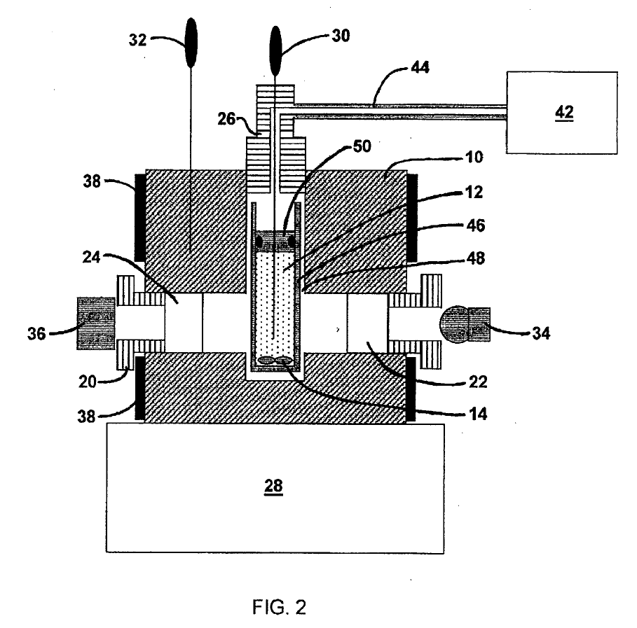

FIG. 2 illustrates another embodiment of the apparatus comprising a variable-volume isolation vial 46. In FIG. 2 the same reference numerals are used to indicate the same features as those previously described with respect to the apparatus depicted in FIG. 1. Use of the variable-volume isolation vial 46 provides an alternative means of containing and pressurizing the brine sample 12. In this embodiment, a pressurization fluid 48 surrounds the vial 46 and forces an O-ring plunger 50 downward until the pressure of the interior of the vial 46 equals the pressure of the pressurization fluid 48 surrounding the vial 46. The vial 46 positioned in close proximity to the surrounding cell 10 creates a gap that surrounds the exterior of the vial 46. The gap is filled with the pressurization fluid 48, originating from the pressure source 42, via the pressurized line 44. Suitable pressurization fluid 48 includes glycol, oil, or other optically clear fluid that will not significantly interfere with the light traversing the cell 10 from the light source 34 to the light detector 36. Likewise, the variable-volume isolation vial 46 may be made of glass, acrylic, polycarbonate, or other optically clear material that can withstand the desired testing temperatures. Containing the entire brine sample 12 within the variable-volume isolation vial 46 advantageously eliminates any concern of the brine sample 12 corroding the interior central cavity of the cell 10 made of metal.

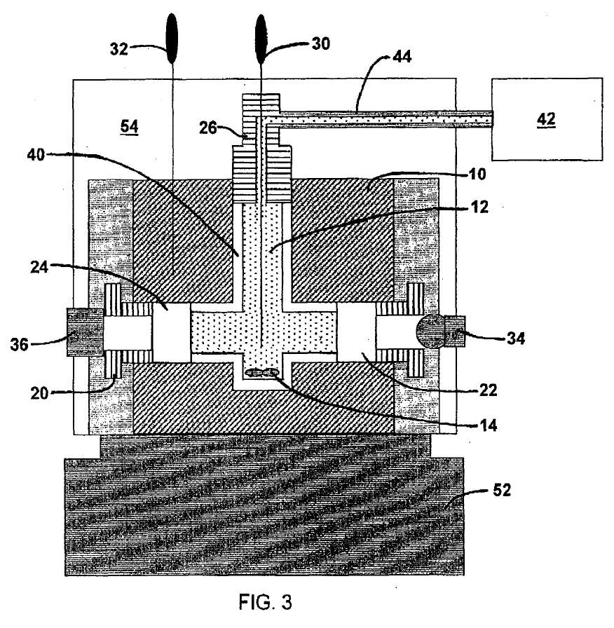

FIG. 3 illustrates another embodiment of the present invention. In FIG. 3 the same reference numerals are used to indicate the same features as those previously described with respect to the apparatus depicted in FIG. 1. In this embodiment, both cooling and stirring of the brine sample 12 is achieved using a cooling magnetic stir-plate 52. Cooling magnetic stir-plates are available commercially from, for example, Digital Stir Kool (model 21485). As depicted, the cell 10 contacts the top surface (plate) of the cooling magnetic stir-plate 52 for cooling. Optionally, to facilitate heat transfer, a heat-transfer grease or oil (not shown) may be used at the interface between the stir-plate 52 and the cell 10. An insulating jacket 54 surrounds the cell 10 and cap 26 to enhance temperature uniformity throughout the cell 10 and brine sample 12 therein. The insulating jacket 54 may be made of any non-conductive material, such as fiberglass, foam or other polymeric material. Similarly, in still another embodiment, the cooling magnetic stir-plate 52 may be used in lieu of the Peltier junctions 38 and the magnetic stirring plate 28 shown in FIG. 2 to provide sufficient cooling and stirring of the brine sample 12 during testing. The use of a cooling magnetic stir-plate 52 as the cooling source has the added advantage of contributing to the portability of the apparatus for on-site or field use.

The method of the present invention includes optically detecting the degree of crystallization of a fluid sample for enhancing the accuracy of the crystallization temperature measurements made in accordance with ANSI/API Recommended Practice 13J, as well as, for automating the test procedure after the initial set-up of the test. Furthermore, the fluid sample may be pressurized using the apparatus of the present invention to determine the crystallization temperatures and the volume reduction of the fluid sample under pressurized conditions. During the initial set-up, the brine fluid sample 12 is poured into the cell 10 containing a small magnetic stir-pill 14 therein. The cell 10 is covered with cap 26, and temperature sensor 30 (e.g., thermocouple) is positioned into the brine sample 12 to continuously measure its temperature. Afterwards, air is purged from the pressurization line 44 connecting the pressure source 42 to the interior of the cell 10, and the pressurization line 44 is filled with brine fluid. The light source 34 is activated to form a light beam that travels in a single pass through the cell 10, and a portion of the brine sample therein, and then into the light detector 36. The computer control software is initialized by entering the desired cooling and heating rates, the desired testing pressure, the set-point hold temperature above LCTD, and the light detector's crystallization event triggering levels. After the initial set-up, the remainder of the test procedure is fully automated.

During the automated cooling/heating test, the crystallization profile of the brine sample 12 is monitored by temperature measurement, volume change measurement, and attenuated light measurement. Upon starting the test, the brine sample 12 is pressurized to the desired constant testing pressure, and subsequently cooled at the set cooling rate until the onset of crystallization is detected by an attenuation in the light as measured by the light detector 36. The minimum temperature reached is recorded as the crystallization event FCTA. At this point, cooling is stopped and the temperature held constant while the exothermic crystallization event causes the brine sample 12 temperature to rise. The maximum temperature reached is recorded as the TCT. Subsequently, the temperature is allowed to fall to an intermediate temperature equal to about one-half the difference between TCT and FCTA, and then the brine sample 12 is heated at the set heating rate. Once the LCTD is detected by the light detector as brine sample clarity, i.e., corresponding to a full strength light beam, the LCTD temperature is recorded, and the brine sample 12 is heated to the set-point hold temperature above LCTD. After a preset hold time, the automated test may be repeated a specified number of times to verify the FCTA, TCT, and LCTD measurements. The automated test may then be executed at the next desired testing pressure. In this manner, the automated apparatus of the present invention may be used to provide highly accurate and reproducible FCTA, TCT, and LCTD temperature measurements of pressurized brine.

While the foregoing is directed to embodiments of the present invention, other and further embodiments of the invention may be devised without departing from the basic scope thereof, and the scope thereof is determined by the claims that follow.

What is claimed is:

1. A pressurized crystallization point test apparatus comprising:
a cell for containing a fluid sample in an interior of the cell, wherein the cell comprises a first transparent window and a second transparent window;
a cooling source for cooling the fluid sample;
a pressure source for pressurizing the fluid sample;
a temperature sensor positioned in the interior of the cell for measuring the temperature of the fluid sample;
an external light source configured to direct light into the cell through the first transparent window; and
an external light detector configured to measure the amount of light that traverses the first transparent window, a portion of the fluid sample, and the second transparent window.

2. The pressurized crystallization point test apparatus of claim 1 further comprising a stirrer positioned in the interior of the cell for circulating the fluid sample.

3. The pressurized crystallization point test apparatus of claim 1, wherein the cooling source is selected from the group consisting of a Peltier junction and a cooling magnetic stir-plate.

4. The pressurized crystallization point test apparatus of claim 1, wherein the external light source is selected from the group consisting of a laser, lamp, and LED.

5. The pressurized crystallization point test apparatus of claim 1, wherein the external light source is positioned outside the cell adjacent the first transparent window, and wherein the external light detector is positioned outside the cell adjacent the second transparent window in a configuration such that light emanating from the external light source may travel in a single pass from the external light source to the external light detector.

6. The pressurized crystallization point test apparatus of claim 1, wherein the portion of the fluid sample that light traverses has a volume of at least about 2 ml.

7. The pressurized crystallization point test apparatus of claim 1, wherein the cell further comprises a liner.

8. The pressurized crystallization point test apparatus of claim 1 further comprising a computer for monitoring the cooling source, the pressure source, the temperature sensor, and the external light detector.

9. A pressurized crystallization point test apparatus comprising:
a variable-volume isolation vial for containing a fluid sample in the interior of the vial, wherein the variable-volume isolation vial is light permeable;
a cell for containing the variable-volume isolation vial within the interior of the cell, wherein the cell comprises a first transparent window and a second transparent window;
a cooling source for cooling the fluid sample;
a pressure source for pressurizing the fluid sample;
a temperature sensor positioned in the interior of the variable-volume isolation vial for measuring the temperature of the fluid sample;
an external light source configured to direct light into the cell through the first transparent window; and
an external light detector configured to measure the amount of light that traverses the first transparent window, a portion of the fluid sample, and the second transparent window.

10. The pressurized crystallization point test apparatus of claim 9 further comprising a stirrer positioned in the interior of the variable-volume isolation vial for circulating the fluid sample.

11. The pressurized crystallization point test apparatus of claim 9 further comprising a pressurization fluid adjacent the external surface of the variable-volume isolation vial for pressurizing the fluid sample therein.

12. The pressurized crystallization point test apparatus of claim 11, wherein the pressurization fluid has a pressure in the range from about 0 psi to about 20,000 psi.

13. The pressurized crystallization point test apparatus of claim 9, wherein the external light source is positioned outside the cell adjacent the first transparent window, and wherein the external light detector is positioned outside the cell adjacent the second transparent window in a configuration such that light emanating from the external light source may travel in a single pass from the external light source to the external light detector.

14. The pressurized crystallization point test apparatus of claim 9, wherein the portion of the fluid sample that light traverses has a volume in the range from about 4 ml to about 10 ml.

15. A method of determining a crystallization temperature of a pressurized fluid, comprising:
providing a cell containing a fluid sample, wherein the cell comprises a first transparent window and a second transparent window;
positioning a temperature sensor into the fluid sample for measuring the temperature of the fluid sample;
pressurizing the fluid sample with a pressure source;
directing a beam of light into the cell, wherein the beam of light originates from a light source positioned outside the cell;
optically detecting the degree of crystallization in the fluid sample by measuring the amount of the beam of light that travels through a portion of the fluid sample and into a light detector, wherein the light detector is positioned outside the cell; and
cooling the fluid sample with a cooling source.

16. The method of claim 15, wherein the pressurizing the fluid sample comprises pressurizing the fluid sample to a pressure in the range from about 0 psi to about 20,000 psi.

17. The method of claim 15, wherein the providing a cell containing a fluid sample comprises positioning a variable-volume isolation vial containing the fluid sample into the interior of the cell, wherein the variable-volume isolation vial is light permeable.

18. The method of claim 17, wherein the pressurizing comprises using a pressurization fluid adjacent the external surface of the variable-volume isolation vial to pressurize the fluid sample therein.

19. The method of claim 15, wherein the portion of the fluid sample that light travels through has a volume of at least about 4 ml.

20. The method of claim 15, further comprising using a computer to automate the cooling of the fluid sample by monitoring with the computer the degree of crystallization optically detected by the light detector.

21. The method of claim 15, further comprising:
optically detecting the onset of crystal formation in the fluid sample while measuring the corresponding temperature;
stopping the cooling of the fluid sample immediately after the onset of crystal formation;
optically detecting the progression of crystal formation in the fluid sample while measuring the corresponding temperature;
heating the fluid sample; and
optically detecting the dissolution of crystals in the fluid sample while measuring the corresponding temperature.

22. The method of claim 21, wherein the measuring the corresponding temperature comprises recording at least one temperature in the group consisting of FCTA, TCT, and LCTD.

23. The method of claim 21, further comprising using a computer to automate the cooling and heating of the fluid sample by monitoring with the computer the temperature of the fluid sample measured by the temperature sensor and the degree of crystallization optically detected by the light detector.
